(19) United States
(12) Patent Application Publication
(10) Pub. No.: US 2025/0003937 A1
(43) Pub. Date: Jan. 2, 2025

(54) Fire-Detecting Sensor Module and Electric Switchboard with the Same

(71) Applicant: HAEMEEL Co.,Ltd, Gyeonggi-do (KR)

(72) Inventor: Haksang YU, Gyeonggi-do (KR)

(21) Appl. No.: 18/679,575

(22) Filed: May 31, 2024

(30) Foreign Application Priority Data
Jun. 28, 2023 (KR) 10-2023-0083854

(51) Int. Cl. G01N 33/00; G08B 17/117; H02B 1/56

(52) U.S. Cl. CPC G01N 33/0067; G01N 33/0031; G01N 33/004; G01N 33/0047; G01N 33/0075; G08B 17/117; H02B 1/56

(57) ABSTRACT

The present invention relates to a fire detection sensor module that is installed inside a switchboard or communication server and can quickly detect fire outbreaks caused by an electric leak or short circuit in power equipment or components.

### TECHNICAL FIELD

The present invention relates to a fire detecting sensor module and an electric switchboard with the same, and more particularly, to a fire detection sensor module that is installed inside a switchboard or communication server and can quickly detect a fire caused by an electric leak or short circuit in power equipment or components.

### BACKGROUND ART

Electrical fires are on the rise with the increase in the use of electrical devices and equipment. This causes not only casualties and property damage but also great damage to the national economy. Therefore, it is important to prevent fires through early detection of electrical fires.

Distribution boards or electric control boards (ex, the electric switchboard, communication server) have the function of receiving and distributing power, and electrical fires cause a lot of damage. In general, the main causes of electrical fires in such switchboards or electrical facilities include short-circuiting of wire cables, overcurrent, arcing, and leakage current.

To reduce the occurrence of such electrical fires, individual equipment such as fire detectors and circuit breakers are installed. Recently, a system has been established that individually installs contact temperature sensors in locations where fire may occur and notifies managers when heat generation is detected through them.

Republic of Korea Patent No. 10-1411958 discloses a distribution board that activates an alarm and an alarm light to provide an alarm when the internal temperature of the distribution board rises above 50° C. When the internal temperature of the distribution board rises above 72° C., the nozzle of the fire extinguisher is automatically opened regardless of whether a fire occurs, so that the fire extinguishing agent can be dispersed into the distribution board.

However, as in the above registered patent, if the internal temperature of the distribution panel rises above 50° C., the ignition source such as the wire has already caught fire and the flame has progressed considerably, or depending on the environment of the installation site or work environment, a fire may not occur even if it is above 50° C. In particular, with the temperature sensor installed inside the switchboard as in the above registered patent, it is very difficult to detect abnormal situations in the heating stage of combustibles (wire coating, etc.) by a specific heat source (short circuit, overcurrent, arc, etc.) before flame generation (ignition).

### DISCLOSURE

### Technical Problem

An object of the present invention is to provide a fire detection sensor module for an electric switchboard that can detect abnormal situations in the heating stage of combustibles (wire coating, etc.) before flame generation (ignition).

Another object of the present invention is to provide a fire detection sensor module that is installed inside a switchboard or communication server and can quickly detect a fire caused by an electric leak or short circuit.

### Technical Solution

An aspect of the present invention provides a fire-detecting sensor module installed inside of an electric switchboard, comprising:

the first sensor unit installed on the upper side of the inside of electric switchboard, comprising of a frame body, a methane gas sensor and/or a carbon monoxide sensor, and a connection terminal to detect the concentration of methane gas and/or carbon monoxide at a predetermined interval; and

a control unit to predict fire outbreaks by receiving gas concentrations data from the first sensor unit.

Another aspect of the present invention provides an electric switchboard capable of detecting fire outbreaks, comprising:

a housing with an air inlet port at the bottom side and an air outlet port at the top side;

a forced ventilation fan installed in the air inlet port or the air outlet port;

an electrical component installed inside the housing;

the first sensor unit installed on the upper side of the inside of the housing, comprising of a frame body, a methane gas sensor and/or a carbon monoxide sensor, and a connection terminal to detect the concentration of methane gas and/or carbon monoxide at a predetermined interval; and

a control unit to predict fire outbreaks by receiving gas concentrations data from the first sensor unit.

### Advantageous Effects

The fire detection sensor module of the present invention can measure the change in concentration of the small amount of methane gas (or carbon monoxide) that is generated before ignition (flame generation), such as when heat is applied to polymers like PE or PP (used in wires or electrical components). As such, it can quickly predict fire outbreaks in the pre-ignition stage. The fire detecting sensor module can spot abnormal situations where wires or electrical components are locally heated, even if the temperature inside the distribution board does not rise significantly (difference of around 2-3° C.). It can therefore prevent not only fires, but also malfunctions or failures of components due to power outages or communication errors.

In addition, the fire detection sensor module of the present invention improves the reliability of the sensor module measuring the concentration of methane gas (or carbon monoxide) on the air inlet side and the air outlet side of the electric switchboard such as distribution boards and communication devices (hereinafter referred to as “electric switchboard”).

### DESCRIPTION OF DRAWINGS

FIG. 1 is a schematic diagram of the electric switchboard, including the fire detection sensor module of the invention.

FIG. 2 is a perspective view of the fire detection sensor module of the invention.

FIG. 3 is a block diagram of the fire detection sensor module of the invention.

FIG. 4 is a conceptual diagram of fire detection system, including the sensor module.

FIGS. 5 and 6 show the trend lines of the data in Table 2.

### MODES OF THE INVENTION

Hereinafter, embodiments and Examples of the present invention will be described in detail so as to easily implement those skilled in the art.

Terms used herein are used only to describe specific embodiments, and are not intended to limit the present invention. A singular form may include a plural form unless otherwise clearly opposed in the context. In the present invention, it should be understood that the term “comprising” or “having” indicates that a feature, a number, a step, an operation, a component, a part or a combination thereof described in the specification is present, but does not exclude a possibility of presence or addition of one or more other features, numbers, steps, operations, components, parts or combinations thereof, in advance.

Additionally, terms such as “unit,” and “module” used in the specification refer to a unit that processes at least one function or operation, and also may refer to hardware, software, or a combination of hardware and software.

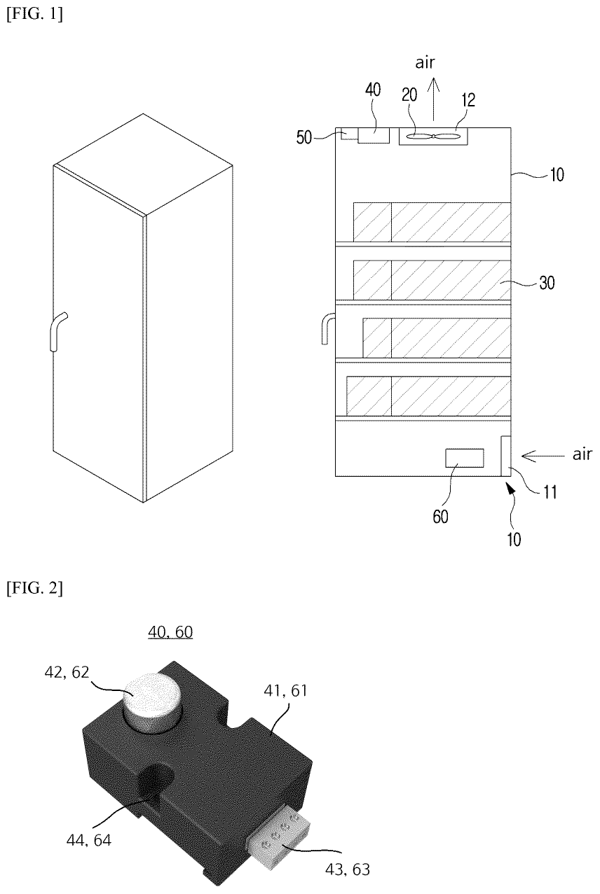

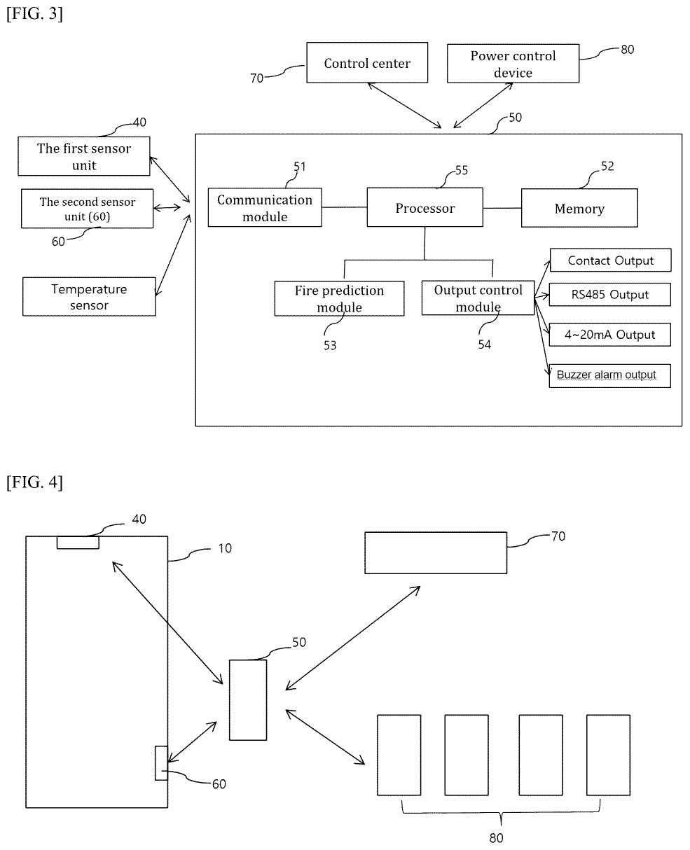

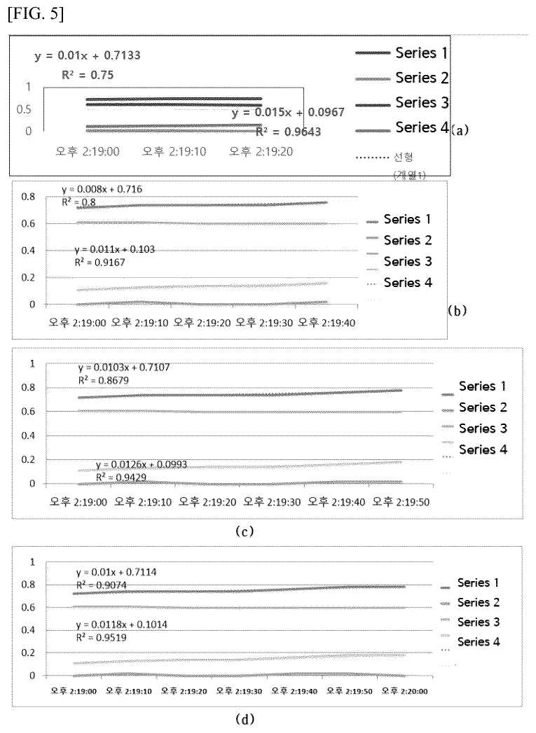

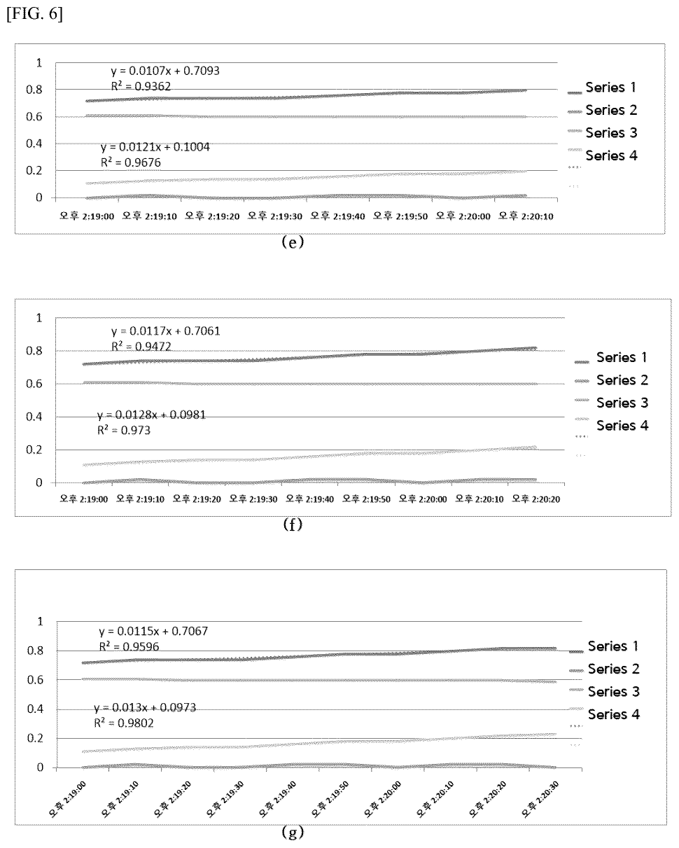

FIG. 1 is a schematic diagram of the electric switchboard, including the fire detection sensor module of the invention, FIG. 2 is a perspective view of the fire detection sensor module of the invention, FIG. 3 is a block diagram of the fire detection sensor module of the invention, FIG. 4 is a conceptual diagram of fire detection system, including the sensor module, FIGS. 5 and 6 show the trend lines of the data in Table 2.

Referring to the FIGS. 1 and 4, the fire-detecting sensor module of the present invention installed inside of an electric switchboard comprises the first sensor unit (40) and a control unit (50).

The fire-detecting sensor module of the present invention can also have the second sensor unit (60) that is installed inside the electric switchboard.

The electric switchboard may include a housing (10) a forced ventilation fan (20) and an electrical device or electrical component (30). Examples of the electric switchboard include a distribution board or a communication device but are not limited thereto.

The housing (10) can be formed as a casing with an air inlet port (11) at the bottom side and an air outlet port (12) at the top side.

The forced ventilation fan (20) can be installed in the air inlet port (11) or the air outlet port (12) to supply air into the housing or forcibly discharge air out from the housing.

The electrical device or electrical component (30) can be well-known communication lines (cables), power lines (cables), communication terminals, electrical terminals installed in the electric switchboard.

The First sensor unit (40) can be installed near the air outlet port (12) inside the housing, and the Second sensor unit (60) can be installed near the air inlet port (11).

The First sensor unit (40) comprises a frame body (41), a methane gas sensor or a carbon monoxide sensor (42), and a connection terminal (43) to detect the concentration of methane gas and/or carbon monoxide at a predetermined interval.

The Second sensor unit (60) comprises a frame body (61), a methane gas sensor or a carbon monoxide sensor (62), and a connection terminal (63) to detect the concentration of methane gas and/or carbon monoxide at a predetermined interval.

The above methane gas sensor or carbon monoxide sensor (42, 62) and connection terminal (43, 63) can be attached to the frame body (61, 62).

The sensor unit (40, 60) can also include a temperature sensor or a humidity sensor. The above sensor unit (40, 60) can be equipped with a methane gas sensor or a carbon monoxide sensor (42, 62).

The preferable sensor for this invention may be the methane gas sensor. Under the same conditions, the amount of methane generated is greater than carbon monoxide and the change in concentration of methane also precedes the change in carbon monoxide. As such, using a methane gas sensor is better for improved reliability and speed

The sensor unit (40, 60) can be equipped with both a methane gas sensor and a carbon monoxide sensor (42, 62). In this case, the sensor module can determine whether a fire has occurred by combining the concentration of methane gas and carbon monoxide concentration over the same period. By adding the two gas concentrations, the sensor module can determine more reliably whether a fire or an emergency has occurred.

The control unit (50) can receive the gas concentration from the First sensor unit (40) or from both the First sensor unit (40) and the Second sensor unit (60) to predict a fire.

The term “fire outbreak” includes an emergency situation where a fire occurs due to ignition within the device, and also an emergency situation in which the covering of wires or power equipment is abnormally heated by a heat source (short circuit, overcurrent, arc, etc.) even if it does not result in ignition.

The gas concentration may be the methane gas concentration or the sum of the methane gas concentration and the carbon monoxide concentration.

If the gas concentration is just methane gas, the concentration may be the measurements from the First sensor unit, or it may be the difference between the concentration from the First sensor unit and the Second sensor unit.

If the gas concentration is the sum of the methane gas and carbon monoxide concentration, the gas concentration may be the sum of the gases (gas concentration) measured by the First sensor unit, or it may be the difference between the sum of the gases measured by the First sensor unit and the Second sensor unit.

The control unit (50) can be installed on the frame body (41) of the sensor unit (40), or on a PC, tablet, as well as another device located outside of the housing.

The control unit (50) includes a communication module (51), memory (52), fire prediction module (53), output control module (54) and processor (55). The fire prediction module (53) and output control module (54) can be implemented as software programs that are executed by the processor (55).

The fire prediction module (53) can be used in many ways to detect a fire.

The fire prediction module (53) is configured to receive the gas concentration at regular intervals and to store it as data of (time, concentration). The received time period may be within 10 minutes, preferably within 1 minute, and more preferably between 1 second and 1 minute. The fire prediction module (53) may calculate the change rate of gas concentration (the current gas concentration compared to the previous gas concentration), and determine the fire outbreaks when the change rate of gas concentration is positive for a certain number of consecutive times.

Table 1 below shows the methane gas and carbon monoxide concentration data from the sensor module attached to the upper and lower parts of the distribution board. The electrical cable was heated using a heat gun from 2:17:00 PM onwards.

Table 2 below shows the concentration change rate using data from Table 1.

TABLE 1

| | upper | bottom | upper | bottom | Upper | |
|---|---|---|---|---|---|---|
| | CH4 | CH4 | CO | CO | CH4 + CO | |
| Time | concentration | concentration | concentration | concentration | concentration | note |
| 2:16:50 | 0.7 | 0.63 | 0.42 | 0.42 | 1.12 | |
| 2:17:00 | 0.7 | 0.63 | 0.42 | 0.42 | 1.12 | HEATING |
| 2:17:10 | 0.7 | 0.63 | 0.42 | 0.42 | 1.12 | |
| 2:17:20 | 0.7 | 0.63 | 0.42 | 0.42 | 1.12 | |
| 2:17:30 | 0.7 | 0.62 | 0.42 | 0.42 | 1.12 | |
| 2:17:40 | 0.7 | 0.62 | 0.42 | 0.42 | 1.12 | |
| 2:17:50 | 0.7 | 0.62 | 0.42 | 0.42 | 1.12 | |
| 2:18:00 | 0.7 | 0.62 | 0.42 | 0.41 | 1.12 | |
| 2:18:10 | 0.7 | 0.62 | 0.42 | 0.41 | 1.12 | |
| 2:18:20 | 0.7 | 0.62 | 0.42 | 0.41 | 1.12 | |
| 2:18:30 | 0.7 | 0.61 | 0.42 | 0.41 | 1.12 | |
| 2:18:40 | 0.7 | 0.61 | 0.42 | 0.41 | 1.12 | |
| 2:18:50 | 0.7 | 0.61 | 0.42 | 0.41 | 1.14 | |
| 2:19:00 | 0.72 | 0.61 | 0.42 | 0.41 | 1.14 | |
| 2:19:10 | 0.74 | 0.61 | 0.42 | 0.41 | 1.18 | |
| 2:19:20 | 0.74 | 0.6 | 0.42 | 0.41 | 1.18 | |
| 2:19:30 | 0.74 | 0.6 | 0.44 | 0.4 | 1.18 | |
| 2:19:40 | 0.76 | 0.6 | 0.44 | 0.4 | 1.2 | |
| 2:19:50 | 0.78 | 0.6 | 0.46 | 0.4 | 1.24 | |
| 2:20:00 | 0.78 | 0.6 | 0.46 | 0.4 | 1.24 | |
| 2:20:10 | 0.8 | 0.6 | 0.46 | 0.4 | 1.26 | |
| 2:20:20 | 0.82 | 0.6 | 0.46 | 0.4 | 1.28 | |
| 2:20:30 | 0.82 | 0.59 | 0.48 | 0.4 | 1.3 | |
| 2:20:40 | 0.82 | 0.59 | 0.48 | 0.4 | 1.3 | |
| 2:20:50 | 0.86 | 0.59 | 0.5 | 0.4 | 1.36 | |
| 2:21:00 | 0.86 | 0.58 | 0.5 | 0.4 | 1.36 | |
| 2:21:10 | 0.9 | 0.58 | 0.5 | 0.4 | 1.4 | |
| 2:21:20 | 0.9 | 0.58 | 0.52 | 0.4 | 1.42 | |
| 2:21:30 | 0.92 | 0.58 | 0.52 | 0.4 | 1.44 | |
| 2:21:40 | 0.94 | 0.58 | 0.54 | 0.4 | 1.48 | |
| 2:21:50 | 0.96 | 0.58 | 0.54 | 0.4 | 1.5 | |
| 2:22:00 | 0.98 | 0.58 | 0.54 | 0.4 | 1.52 | |

TABLE 2

| | upper | Change | bottom | Concentration | | |
|---|---|---|---|---|---|---|
| | CH4 | rate of | CH4 | difference of | | |
| Time | concentration | concentration | concentration | CH4 | Data denote | note |
| 2:16:50 | 0.7 | 0 | 0.63 | 0.07 | | |
| 2:17:00 | 0.7 | 0 | 0.63 | 0.07 | | heating |
| 2:17:10 | 0.7 | 0 | 0.63 | 0.07 | | |
| 2:17:20 | 0.7 | 0 | 0.63 | 0.07 | | |
| 2:17:30 | 0.7 | 0 | 0.62 | 0.08 | | |
| 2:17:40 | 0.7 | 0 | 0.62 | 0.08 | | |
| 2:17:50 | 0.7 | 0 | 0.62 | 0.08 | | |
| 2:18:00 | 0.7 | 0 | 0.62 | 0.08 | | |
| 2:18:10 | 0.7 | 0 | 0.62 | 0.08 | | |
| 2:18:20 | 0.7 | 0 | 0.62 | 0.08 | | |
| 2:18:30 | 0.7 | 0 | 0.61 | 0.09 | | |
| 2:18:40 | 0.7 | 0 | 0.61 | 0.09 | | |
| 2:18:50 | 0.7 | 0 | 0.61 | 0.11 | | |
| 2:19:00 | 0.72 | 0 | 0.61 | 0.11 | D1 | Reference |
| 2:19:10 | 0.74 | 0.02 | 0.61 | 0.13 | D2 | |
| 2:19:20 | 0.74 | 0 | 0.6 | 0.14 | D3 | |
| 2:19:30 | 0.74 | 0 | 0.6 | 0.14 | D4 | |
| 2:19:40 | 0.76 | 0.02 | 0.6 | 0.16 | D5 | |
| 2:19:50 | 0.78 | 0.02 | 0.6 | 0.18 | D6 | |
| 2:20:00 | 0.78 | 0 | 0.6 | 0.18 | D7 | |
| 2:20:10 | 0.8 | 0.02 | 0.6 | 0.2 | D8 | |
| 2:20:20 | 0.82 | 0.02 | 0.6 | 0.22 | D9 | |
| 2:20:30 | 0.82 | 0 | 0.59 | 0.23 | D10 | |
| 2:20:40 | 0.82 | 0 | 0.59 | 0.23 | D11 | |
| 2:20:50 | 0.86 | 0.04 | 0.59 | 0.27 | D12 | |
| 2:21:00 | 0.86 | 0 | 0.58 | 0.28 | | |
| 2:21:10 | 0.9 | 0.04 | 0.58 | 0.32 | | |
| 2:21:20 | 0.9 | 0 | 0.58 | 0.32 | | |
| 2:21:30 | 0.92 | 0.02 | 0.58 | 0.34 | | |
| 2:21:40 | 0.94 | 0.02 | 0.58 | 0.36 | | |
| 2:21:50 | 0.96 | 0.02 | 0.58 | 0.38 | | |
| 2:22:00 | 0.98 | 0.02 | 0.58 | 0.4 | | |

The change rate of gas concentration is calculated by dividing the difference between the current concentration and the previous concentration by the time difference. For instance, the change rate can be expressed as Change Rate=(C−(C−1))/(t−(t−1)), where t is the current time, (t−1) is the previous time, C is the gas concentration at time t and (C−1) is the gas concentration at time (t−1).

For instance, if the change rate of gas concentration is positive for four or more consecutive times, the fire prediction module (53) can determine that the fire outbreaks is occurring. The specific number of consecutive positive change rates can be adjusted based on factors such as the measurement interval.

Additionally, the fire prediction module (53) can also determine a fire using the method below.

The fire prediction module (53) is configured to receive the gas concentrations at regular intervals, and store them as a data of (time, gas concentration).

The fire prediction module (53) can calculate the change rate of the gas concentration (the current gas concentration compared to the previous gas concentration). If there is data (time, gas concentration) whose change rate converted to a positive value among the sequentially received data (time, concentration), the data can be selected as reference data D1 (t_{D1}, C_{D1}). Alternatively, the control unit may select the previous data as reference data, wherein the previous data precedes the data whose change rate converted to a positive value.

The fire prediction module (53) can store (time, concentration) data sequentially received after the reference data D1 (t_{D1}, C_{D1}) as D2 (t_{D2}, C_{D2}) to Dn (t_{Dn}, C_{Dn}), wherein, n is an integer of 2 or more.

The fire prediction module (53) may determine the time at Dn (t_{Dn}, C_{Dn}) as time of the fire outbreaks, when Dn (tDn, CDn) has a gas concentration value that increases beyond a predetermined range compared to the gas concentration of the reference data D1 (t_{D1}, C_{D1}).

Based on Table 2, FIGS. 5 and 6, for example, the fire prediction module (53) can identify the fire outbreaks if there is a 10˜20% increase compared to the gas concentration of the reference data D1 (t_{D1}, C_{D1}).

Additionally, the fire prediction module (53) is configured to receive the gas concentrations at regular intervals, and store them as a data of (time, gas concentration).

The fire prediction module (53) may calculate the change rate of the current gas concentration compared to the previous gas concentration. If there is data (time, gas concentration) whose change rate converted to a positive value among the sequentially received data (time, concentration), the data can be selected as reference data D1 (t_{D1}, C_{D1}). Alternatively, the control unit may select the previous data as reference data, wherein the previous data precedes the data whose change rate converted to a positive value.

The fire prediction module (53) may calculate the gas concentration difference between the first sensor unit and the second sensor unit and store them as data of (time, concentration difference). The fire prediction module (53) may denote and store the data of (time, concentration difference) sequentially received after the reference data D1 (t_{D1}, C_{D1}). as D2 (tD2, CD2 concentration difference) to Dn (tDn, CDn concentration difference).

The fire prediction module (53) may determine the time at Dn (tDn, CDn concentration difference) as time of the fire outbreaks, when Dn (tDn, CDn concentration difference) has a gas concentration difference value that increases beyond a predetermined range compared to the gas concentration difference of the reference data D1 (tD1, CD1). For example, the predetermined range can be 1.5 to 2 times.

Referring to the Table 2, the fire prediction module (53) can determine the fire outbreaks if the gas concentration difference between the first sensor unit (40) and the second sensor unit (60) (upper-lower concentration difference) is 1.5 to 2 times the baseline concentration.

As seen in Table 2, Concentration difference of CH4 means the difference between the upper CH4 concentration and the lower CH4 concentration. The upper CH4 concentration is measured by the first sensor unit (40), and the lower CH4 concentration is measured by the second sensor unit 60.

Referring to the Table 2, the CD1 concentration difference which is the difference in upper-lower concentration in reference data D1 is 0.11. The concentration difference of D9 is 0.22. Since the concentration difference at D9 is twice the concentration difference at D1, the fire prediction module (53) can determine the time (2:20:20) at D9 as the fire outbreaks time and activate a fire alarm.

The CH4 concentration difference between the first sensor unit (40) and the second sensor unit (60) reveals a significantly higher gas concentration change rate compared to using the methane gas concentration measured solely by the First sensor unit (40).). This indicates that employing both the First sensor unit (40) and the Second sensor unit (60) together results in a higher gas concentration change rate, enabling faster fire detection compared to just using the First sensor unit (40) alone.

Additionally, the fire prediction module (53) is configured to receive the gas concentrations at regular intervals, and store them as a data of (time, gas concentration).

The fire prediction module (53) can calculate the change rate of the gas concentration (the current gas concentration compared to the previous gas concentration). If there is data (time, gas concentration) whose change rate converted to a positive value among the sequentially received data (time, concentration), the data can be selected as reference data D1 (t_{D1}, C_{D1}). Alternatively, the control unit may select the previous data as reference data, wherein the previous data precedes the data whose change rate converted to a positive value.

The fire prediction module (53) can store (time, concentration) data sequentially received after the reference data D1 (t_{D1}, C_{D1}) as D2 (t_{D2}, C_{D2}) to Dn (t_{Dn}, C_{Dn}), wherein, n is an integer of 2 or more.

The fire prediction module (53) may calculate the trend line of Dn (tDn, CDn) from the reference data D1 (tD1, CD1), respectively.

The trend line from Table 2 data is shown in FIGS. 5 and 6.

D2 trendline can be obtained using regression analysis based on the coordinates D1 (tD1, CD1) and D2 (tD2 CD2), and D5 trendline can be obtained using regression analysis based on the coordinates D1 (tD1, CD1), D2 (tD2 CD2), D3 (Td3, CD3), D4 (tD4 CD5) and D5 (tD5, CD5).

Dn trendline can be obtained using regression analysis based on the coordinates D1 (tD1, CD1), D2 (tD2 CD2), D3 (Td3, CD3) . . . Dn (tDn CDn).

From Table 2 and FIGS. 5 and 6, we can see that (a) represents the trendline for D3, (b) represents the trendline for D5, (c) represents the trendline for D6, (d) represents the trendline for D7, (e) represents the trendline for D8, (f) represents the trendline for D9 and (g) represents the trendline for D10.

FIG. 6 and its trendline graph show that the upper curve series (Series 1) represents the methane gas concentration on the upper side, while Series 2 represents the methane gas concentration on the lower side. Series 3 represents the change rate of methane gas concentration on the upper side and Series 4 represents the difference in methane gas concentration between the upper and lower sides over time.

The fire prediction module (53) may determine the time at Dn (tDn, CDn) as time of the fire outbreaks, when the variance value (R2) of the trend line of Dn (tDn, CDn) is greater than the predetermined variance value set, wherein, n is 5 or higher. If n is 4 or less, the number of data is small and prediction reliability may be reduced.

The predetermined variance value can be set in the range of 0.95 to 0.99. When the variance value is preferably 0.97 to 0.99, prediction reliability can be increased.

The predetermined variance value may vary depending on factors like time intervals.

Referring to the trendline for the upper methane gas series (Series 1) and Table 2, the data with a trendline that has an variance value of 0.95 or higher is D9 (2:20:20). Therefore, the fire prediction module (53) can determine the fire outbreak and trigger an alarm at 2:20:20.

From FIGS. 5 and 6, the graph of Series 4 represents the trendline of the CH4 concentration difference between the upper CH4 concentration and the lower CH4 concentration, the data with a trendline that has an variance value of 0.95 or higher is D7 (2:20:00). Therefore, the fire prediction module (53) can determine the fire outbreak and trigger an alarm at 2:20:00.

As seen above, this indicates that employing both the First sensor unit (40) and the Second sensor unit (60) together results in a higher gas concentration change rate, enabling faster fire detection compared to just using the First sensor unit (40) alone.

Meanwhile, the fire prediction module (53) can remove data without storing it in the memory 52 where the gas concentration change rate has a negative value. Alternatively, the fire prediction module (53) may remove the previous data, wherein the previous data precedes the data whose change rate is the negative value.

Table 3 represents the gas concentration measurements obtained from the sensor unit during a sequence of events in the electric switchboard (FIG. 6): wire heating, heating termination, and wire reheating.

[symbol]

[symbol]

3

[symbol]

| | upper | Change rate | bottom | Concentration | |
|---|---|---|---|---|---|
| | CH4 | of | CH4 | difference of | |
| Time | concentration | concentration | concentration | CH4 | Data denote |
| PM 5:16:50 | 0.7 | 0 | 0.63 | 0.07 | |
| 5:17:00 | 0.7 | 0 | 0.63 | 0.07 | |
| 5:17:10 | 0.7 | 0 | 0.63 | 0.07 | |
| 5:17:20 | 0.7 | 0 | 0.63 | 0.07 | |
| 5:17:30 | 0.7 | 0 | 0.62 | 0.08 | |
| 5:17:40 | 0.7 | 0 | 0.62 | 0.08 | heating |
| 5:17:50 | 0.7 | 0 | 0.62 | 0.08 | |
| 5:18:00 | 0.7 | 0 | 0.62 | 0.08 | |
| 5:18:10 | 0.7 | 0 | 0.62 | 0.08 | |
| 5:18:20 | 0.7 | 0 | 0.62 | 0.08 | |
| 5:18:30 | 0.7 | 0 | 0.61 | 0.09 | |
| 5:18:40 | 0.7 | 0 | 0.61 | 0.09 | D1 |
| 5:18:50 | 0.72 | 0.02 | 0.61 | 0.11 | D2 |
| 5:19:00 | 0.72 | 0 | 0.61 | 0.11 | D3 |
| 5:19:10 | 0.74 | 0.02 | 0.61 | 0.13 | D4 |
| 5:19:20 | 0.76 | 0.02 | 0.6 | 0.16 | heating termination |
| | | | | | D5 |
| 5:19:30 | 0.76 | 0 | 0.6 | 0.16 | D6 |
| 5:19:40 | 0.78 | 0.02 | 0.6 | 0.18 | D7 |
| 5:19:50 | 0.78 | 0 | 0.6 | 0.18 | D8 |
| 5:20:00 | 0.76 | −0.02 | 0.6 | 0.16 | D9 |
| | | | | | Remove D1~D9 |
| 5:20:10 | 0.76 | 0 | 0.6 | 0.16 | Reheating |
| 5:20:20 | 0.76 | 0 | 0.6 | 0.16 | |
| 5:20:30 | 0.74 | 0 | 0.59 | 0.15 | |
| 5:20:40 | 0.74 | 0 | 0.59 | 0.15 | |
| 5:20:50 | 0.74 | 0 | 0.59 | 0.17 | |
| 5:21:00 | 0.74 | 0 | 0.58 | 0.16 | |
| 5:21:10 | 0.74 | 0 | 0.58 | 0.16 | D1 |
| 5:21:20 | 0.76 | 0.02 | 0.58 | 0.18 | D2 |
| 5:21:30 | 0.76 | 0 | 0.58 | 0.18 | D3 |
| 5:21:40 | 0.76 | 0 | 0.58 | 0.18 | D4 |
| 5:21:50 | 0.78 | 0.02 | 0.58 | 0.2 | D5 |
| 5:22:00 | 0.78 | 0 | 0.58 | 0.2 | D6 |
| 5:22:10 | 0.82 | 0.04 | 0.58 | 0.24 | D7 |
| 5:22:20 | 0.82 | 0 | 0.57 | 0.25 | D8 |
| 5:22:30 | 0.84 | 0.02 | 0.57 | 0.27 | D9 |
| 5:22:40 | 0.84 | 0 | 0.57 | 0.27 | D10 |
| 5:22:50 | 0.86 | 0.02 | 0.57 | 0.29 | D11 |
| 5:23:00 | 0.86 | 0 | 0.57 | 0.29 | D12 |

According to Table 3, heating started at 5:17:40, and the change rate increased to 0.02 at 5:18:50. Therefore, the reference time can be set to 5:18:40 (D1). However, after heating was interrupted at D5, the gas concentration change rate became negative at D9. In this case, data points (D1˜D9) before D9 can be excluded from the trendline acquisition data.

Additionally, as per Table 3, the change rate rises to 0.02 at 5:21:20. Therefore, the reference time can be re-established to 5:21:10 (D1), and trendlines for the subsequently collected data D2˜D12˜Dn can be obtained using the same method used for the data in Table 2 .

Another aspect of the present invention provides an electric switchboard capable of detecting fire outbreaks.

The electric switchboard comprises a housing (10), a forced ventilation fan (20), an electrical component [30], the first sensor unit (40), a control unit (50) and the second sensor unit (60).

Regarding the housing (10), the forced ventilation fan (20), the electrical component [30], the first sensor unit (40), the control unit (50) and the second sensor unit (60), you can refer to the information explained above.

Another aspect of the present invention provides an a fire-detecting system.

The fire-detecting system comprises a housing (10), a forced ventilation fan (20), an electrical component [30], the first sensor unit (40), a control unit (50), the second sensor unit (60), and a power control device (80).

Regarding the housing (10), the forced ventilation fan (20), the electrical component [30], the first sensor unit (40), the control unit (50) and the second sensor unit (60), you can refer to the information explained above.

The power control device (80) receives signals from the control unit (50) and can either transmit a fire alarm to a control center (70) or cut off the power supply to the equipment in building.

The power control device (80) can be a known EDCR or PLC control device.

As described above, specific embodiments of the present invention have been described. It is understood to those skilled in the art that the present invention may be implemented as modified forms without departing from essential features of the present invention. The scope of the present invention is defined by the appended claims rather than by the foregoing description, and all differences within the scope of equivalents thereof should be construed as being included in the present invention.

1. A fire-detecting sensor module installed inside of electric switchboard, comprising:
the first sensor unit installed on the upper side of the inside of electric switchboard, comprising of a frame body, a methane gas sensor and/or a carbon monoxide sensor, and a connection terminal to detect the concentration of methane gas and/or carbon monoxide at a predetermined interval; and
a control unit to predict fire outbreaks by receiving gas concentrations data from the first sensor unit.

2. The sensor module as claimed in claim 1, wherein the sensor module comprises the second sensor unit installed on the bottom side of the inside of electric switchboard, comprising of a frame body, a methane gas sensor and/or a carbon monoxide sensor, and a connection terminal to detect the concentration of methane gas and/or carbon monoxide at a predetermined interval,
wherein the control unit predicts fire outbreaks by receiving gas concentrations data from the first sensor unit and/or the second sensor unit.

3. The sensor module as claimed in claim 1, wherein the control unit is configured to receive the gas concentration at regular intervals and to store it as data of (time, concentration) and to calculate the change rate of the current gas concentration compared to the previous gas concentration, and to determine the fire outbreaks when the change rate is positive for a certain number of consecutive times.

4. The sensor module as claimed in claim 1, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn), and
to determine the time at Dn (tDn, CDn) as time of the fire outbreaks, when Dn (tDn, CDn) has a gas concentration value that increases beyond a predetermined range compared to the gas concentration of the reference data D1 (tD1, CD1).
Wherein, n is an integer of 2 or more.

5. The sensor module as claimed in claim 1, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to calculate the gas concentration difference between the first sensor unit and the second sensor unit and store them as (time, concentration difference) data
to denote (time, concentration difference) data sequentially received after the reference data D1 (tD1, CD1) as D2 (tD2, CD2 concentration difference) to Dn (tDn, CDn concentration difference), and
to determine the time at Dn (tDn, CDn concentration difference) as time of the fire outbreaks, when Dn (tDn, CD concentration difference) has a gas concentration difference value that increases beyond a predetermined range compared to the gas concentration difference of the reference data D1 (tD1, CD1).
Wherein, n is an integer of 2 or more.

6. The sensor module as claimed in claim 1, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn),
to calculate the trend line of Dn (tDn, CDn) from the reference data D1 (tD1, CD1), respectively, and
to determine the time at Dn (tDn, CDn) as time of the fire outbreaks, when the variance value (R2) of the Dn (tDn, CDn) is greater than the predetermined variance value set.
Wherein, n is an integer of 2 or more.

7. An electric switchboard capable of detecting fire outbreaks, comprising:
a housing with an air inlet port at the bottom side and an air outlet port at the top side;
a forced ventilation fan installed in the air inlet port or the air outlet port;
an electrical component installed inside the housing;
the first sensor unit installed on the upper side of the inside of the housing, comprising of a frame body, a methane gas sensor and/or a carbon monoxide sensor, and a connection terminal to detect the concentration of methane gas and/or carbon monoxide at a predetermined interval; and
a control unit to predict fire outbreaks by receiving gas concentrations data from the first sensor unit.

8. The electric switchboard as claimed in claim 7, wherein the electric switchboard comprises the second sensor unit installed on the bottom side of the inside of electric switchboard, comprising of a frame body, a methane gas sensor and/or a carbon monoxide sensor, and a connection terminal to detect the concentration of methane gas and/or carbon monoxide at a predetermined interval,
wherein the control unit predicts fire outbreaks by receiving gas concentrations data from the first sensor unit and/or the second sensor unit.

9. The electric switchboard as claimed in claim 7, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn), and
to determine the time at Dn (tDn, CDn) as time of the fire outbreaks, when Dn (tDn, CDn) has a gas concentration value that increases beyond a predetermined range compared to the gas concentration of the reference data D1 (tD1, CD1).
Wherein, n is an integer of 2 or more.

10. The electric switchboard as claimed in claim 7, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to calculate the gas concentration difference between the first sensor unit and the second sensor unit and store them as (time, concentration difference) data
to denote (time, concentration difference) data sequentially received after the reference data D1 (tD1, CD1) as D2 (tD2, CD2 concentration difference) to Dn (tDn, CDn concentration difference), and
to determine the time at Dn (tDn, CDn concentration difference) as time of the fire outbreaks, when Dn (tDn, CD concentration difference) has a gas concentration difference value that increases beyond a predetermined range compared to the gas concentration difference of the reference data D1 (tD1, CD1).
Wherein, n is an integer of 2 or more.

11. The electric switchboard as claimed in claim 7, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn),
to calculate the trend line of Dn (tDn, CDn) from the reference data D1 (tD1, CD1), respectively, and
to determine the time at Dn (tDn, CDn) as time of the fire outbreaks, when the variance value (R2) of the Dn (tDn, CDn) is greater than the predetermined variance value set.
Wherein, n is an integer of 2 or more.

12. The sensor module as claimed in claim 2, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to calculate the gas concentration difference between the first sensor unit and the second sensor unit and store them as (time, concentration difference) data
to denote (time, concentration difference) data sequentially received after the reference data D1 (tD1, CD1) as D2 (tD2, CD2 concentration difference) to Dn (tDn, CDn concentration difference), and
to determine the time at Dn (tDn, CDn concentration difference) as time of the fire outbreaks, when Dn (tDn, CD concentration difference) has a gas concentration difference value that increases beyond a predetermined range compared to the gas concentration difference of the reference data D1 (tD1, CD1).
Wherein, n is an integer of 2 or more.

13. The sensor module as claimed in claim 2, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn),
to calculate the trend line of Dn (tDn, CDn) from the reference data D1 (tD1, CD1), respectively, and
to determine the time at Dn (tDn, CDn) as time of the fire outbreaks, when the variance value (R2) of the Dn (tDn, CDn) is greater than the predetermined variance value set.
Wherein, n is an integer of 2 or more.

14. The electric switchboard as claimed in claim 8, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to calculate the gas concentration difference between the first sensor unit and the second sensor unit and store them as (time, concentration difference) data
to denote (time, concentration difference) data sequentially received after the reference data D1 (tD1, CD1) as D2 (tD2, CD2 concentration difference) to Dn (tDn, CDn concentration difference), and
to determine the time at Dn (tDn, CDn concentration difference) as time of the fire outbreaks, when Dn (tDn, CD concentration difference) has a gas concentration difference value that increases beyond a predetermined range compared to the gas concentration difference of the reference data D1 (tD1, CD1).
Wherein, n is an integer of 2 or more.

15. The electric switchboard as claimed in claim 8, wherein the control unit is configured to receive the gas concentrations at regular intervals, and store them as a data of (time, gas concentration),
to calculate the change rate of the current gas concentration compared to the previous gas concentration, when the change rate is positive the control unit selects the (time, concentration) data as reference data D1 (tD1, CD1),
to store (time, concentration) data sequentially received after the reference data D1 (tD1, CD1) as D2 (tD2, CD2) to Dn (tDn, CDn),
to calculate the trend line of Dn (tDn, CDn) from the reference data D1 (tD1, CD1), respectively, and
to determine the time at Dn (tDn, CDn) as time of the fire outbreaks, when the variance value (R2) of the Dn (tDn, CDn) is greater than the predetermined variance value set. Wherein, n is an integer of 2 or more.
